(19) United States
(12) Patent Application Publication
(10) Pub. No.: US 2026/0240578 A1
(43) Pub. Date: Aug. 20, 2026

(54) BONE WOUND TREATMENT SYSTEMS AND METHODS USING A BIOMECHANICAL STABILIZING IMPLANT

(71) Applicant: ThoraGenix Innovations, Inc., Tampa, FL (US)

(72) Inventors: Frederick J. Thabet, Boca Raton, FL (US); Ravi Kanagala, Boca Raton, FL (US); Keri George, Calgary (CA)

(73) Assignee: ThoraGenix Innovations, Inc., Tampa, FL (US)

(21) Appl. No.: 19/338,272

(22) Filed: Sep. 24, 2025

Related U.S. Application Data
(60) Provisional application No. 63/698,441, filed on Sep. 24, 2024.
(60) Provisional application No. 63/735,206, filed on Dec. 17, 2024.

(51) Int. Cl. A61B 17/80

(52) U.S. Cl. CPC A61B 17/8076

(57) ABSTRACT

Systems, methods, and devices include a biomechanical stabilizing implant with features for securing and healing two sections of bone together. The biomechanical stabilizing implant has a particular geometry which optimizes an effectiveness of bone growth agents for adjoining the sections of bone being healed together. For instance, the biomechanical stabilizing implant has a central body portion with a first singular fin extending from a first side and a second singular fin extending from the second side. The singular fin(s) have a curved depth profile and a tapered width profile. A greatest depth value of the curved depth profile is at a same point along a length dimension of the biomechanical stabilizing implant as a greatest width value. In this way the singular fin(s) are deepest and widest at the same length dimension point, and become narrower and shallower at either end of the biomechanical stabilizing implant.

### CROSS-REFERENCE TO RELATED APPLICATIONS

This application claims priority to Provisional Patent Application Ser. No. 63/698,441, filed on Sep. 24, 2024, and titled “BONE WOUND TREATMENT GRAFT SYSTEMS AND METHODS,” and to Provisional Patent Application Ser. No. 63/735,206, filed on Dec. 17, 2024 and titled “BONE WOUND TREATMENT SYSTEMS AND METHODS USING A BIOMECHANICAL STABILIZING IMPLANT,” the entireties of which are incorporated by reference herein.

### BACKGROUND

The sternum is a long, flat bone forming the middle portion of the front of the chest. Individual rib bones are connected along the sides of the sternum via cartilage to form the ribcage/thoracic cage which protects the heart, lungs, and major blood vessels from injury. The sternum is cut open during a sternotomy to gain access to the thoracic contents when performing cardiothoracic surgery.

A sternotomy is a surgical procedure in which a midline longitudinal incision is made through at least a portion of the sternum to allow opposing halves/portions to be laterally separated to provide access to organs within the ribcage/thoracic cage. When the surgical procedure is complete, the separated halves/portions are aligned with and secured to one another and the incision closed.

The ideal goal for assisting with closing and healing of sternotomy wounds is complete rejoining of sternal portions with new bone growth in the absence of complications. Unfortunately, patient recovery from a conventional sternotomy is often slow and problematic. As the two sections (of the sternum) are brought back together by the surgeon, proper compression and a tight realignment of the end plates of the sternal surfaces is rarely achieved resulting in non-union, or dehiscence, of separated sternal halves. This non-union allows for motion such as sliding of the surface of one sternal half against the surface of the other sternal half leading to significant pain for the patient and increased chance for development of infection. Additionally, the lack of proper compression leads to the formation of fibrous scar tissue instead of the desired new bone. If further surgical procedures are required, the scar tissue must be removed, further complicating the procedure.

It is with these observations in mind, among others, that various aspects of the present disclosure were conceived and developed.

### SUMMARY

The systems, methods, and devices disclosed herein address the aforementioned issues. For instance, a method of biomechanically stabilizing a first bone portion and a second bone portion by using an implant can include positioning the implant between the first bone portion and the second bone portion with a first fin extending from a central body portion of the implant. The first fin can have a first outer surface operable to form a first friction fit with an exposed inner area of the first bone portion. Also, a second fin can extend from the central body portion in a direction opposite the first fin. The second fin can have a second outer surface operable to form a second friction fit with an exposed inner area of the second bone portion. Additionally, the method can include compressing the implant between the first bone portion and the second bone portion; and/or securing the first bone portion, the implant, and the second bone portion together under compression to form a biomechanically stabilized sternal construct.

In some examples, the biomechanically stabilized sternal construct can use the first friction fit and the second friction fit to form a friction-based gasket preventing movement of the first bone portion relative to the second bone portion. The movement prevented by the friction-based gasket can include movement caused by a plurality of different forces. For example, the plurality of different forces can include a shear force and/or the friction-based gasket can prevent a sliding movement, caused by the shear force, of the first bone portion relative to the second bone portion. Also, the plurality of different forces can include a torque force and/or the friction-based gasket can prevent a rotational movement, caused by the torque force, of the first bone portion relative to the second bone portion. Additionally, the plurality of different forces can include a tensional force and/or the friction-based gasket can prevent a pulling away movement, caused by the tensional force, of the first bone portion relative to the second bone portion. Furthermore, the plurality of different forces can include a force caused by an expansion or contraction of lungs of a patient receiving the implant. The friction-based gasket can prevent a booking motion of the first bone portion relative to the second bone portion caused by the expansion or the contraction of the lungs. Moreover, the biomechanically stabilized sternal construct can one or more wires and/or one or more plates used to secure the first bone portion, the implant, and the second bone portion together.

In some instances, a method of biomechanically stabilizing a first bone portion and a second bone portion by using an implant can include positioning the implant between the first bone portion and the second bone portion. The implant can include a first extension operable to form a first friction fit with an exposed inner area of the first bone portion. Also, the implant can include a second extension operable to form a second friction fit with an exposed inner area of the second bone portion. Additionally, the method can include securing the first bone portion, the implant, and the second bone portion together under compression and using one or more plates or wires to form a biomechanically stabilized sternal construct.

In some scenarios, the implant can provide at least dual functions as a biomechanical stabilization tool and a healing tool. Moreover, the implant can provide at least dual functions as a biomechanical stabilization tool and a blood loss reduction tool. Additionally, the implant can provide at least dual functions as a biomechanical stabilization tool and a pain reduction tool. Also, the method can include maintaining the compression of the biomechanically stabilized sternal construct with at least one of a wire or a plate coupled to the first bone portion and the second bone portion.

In some examples, a method of biomechanically stabilizing a first bone portion and a second bone portion by using an implant can include positioning the implant between the first bone portion and the second bone portion. The method can also include forming a first friction fit with an exposed inner area of the first bone portion and a first portion of the implant. Furthermore, the method can include forming a second friction fit with an exposed inner area of the second bone portion a second portion of the implant. Additionally, the method can include securing the first bone portion, the implant, and the second bone portion together under compression to form a biomechanically stabilized sternal construct. The biomechanically stabilized sternal construct can prevent movement resulting from a shear force, a torque force, a tension force, and a booking force. Also, the method can include immobilizing, with the biomechanically stabilized sternal construct, the first bone portion relative to the second bone portion in at least three degrees of freedom.

### BRIEF DESCRIPTION OF THE DRAWINGS

FIG. 1 depicts a perspective view of a system including a biomechanical stabilizing implant with one or more fin extensions.

FIG. 2 depicts a front view of a system including a biomechanical stabilizing implant with one or more fin extensions.

FIG. 3 depicts a side view of a system including a biomechanical stabilizing implant with one or more fin extensions.

FIG. 4 depicts a top view of a system including a biomechanical stabilizing implant with one or more fin extensions.

FIG. 5 depicts an example system to heal two portions of bone with a biomechanical stabilizing implant.

FIGS. 6A-6C depict example methods of performing a bone stabilization procedure using the systems depicted in FIGS. 1-5.

### DETAILED DESCRIPTION

It will be appreciated that numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

The systems, methods, and devices disclosed herein include a biomechanical stabilizing implant with features that improve the ability to secure and heal two sections of bone together. The biomechanical stabilizing implant can have a particular geometry which optimizes an effectiveness of bone growth agents for receiving and controlling bone growth for the sections of bone being adjoined while immobilizing the two sections of bone. For instance, the biomechanical stabilizing implant can have a central body portion with a first singular fin extending from a first side and a second singular fin extending from a second side. These fin extensions can have a particular shape including a curved depth profile and a tapered width profile. A greatest depth value of the curved depth profile can be at a same point along a length dimension of the biomechanical stabilizing implant as a greatest width value. In this way the singular fin(s) can be deepest and widest at the same length dimension point, and can become narrower and shallower at either end of the biomechanical stabilizing implant.

These features, among others disclosed herein, can improve the ability to secure the biomechanical stabilizing implant between the two sections of bone. The sternum is the anchoring plate for the thoracic skeletal system and can act as the stabilizing point from which the other bones of the thoracic skeletal system articulate and move. Once the sternum is split, four distinct planes of movement are introduced into the sternum which can include the following forces: torsion, shear, expansion & contraction and distraction (e.g., tensional). These forces can be significantly reduced and minimized by use of the sternal stabilization and union graft disclosed herein. As such, the biomechanical stabilizing implant can strengthen and stabilize the bone sections, reduce shearing, booking, or any other motions of the bone sections relative to each other.

For instance, the biomechanical stabilizing implant disclosed herein can operate as a biomechanical stabilization tool. The biomechanical stabilization can be created due to friction of the sternal edges of the bone sections pressing against the biomechanical stabilizing implant, preventing movement (e.g., similar to a friction-fit gasket). Moreover, the biomechanical stabilization generated by the biomechanical stabilizing implant can create immobilization with respect to a plurality of different forces operating on the biomechanically stabilized sternal construct. In some examples, the biomechanical stabilization can prevent movement due to a shear force, such as a sliding movement of one section of bone relative to each other. Additionally, the biomechanical stabilization can prevent movement due to a torque force, such as a rotational movement of the sections of bone relative to each other. Furthermore, the biomechanical stabilization can prevent movement due to a tensional force, such as a pulling away of one bone section from another. Also, the biomechanical stabilization can prevent movement due to a booking force, which can result from an expansion and/or retraction of the lungs causing the two bone sections to fold relation each other. In other words, the biomechanical stabilization can immobilize the first bone section relative to the second bone section in multiple degrees of freedom, such as a vertical y-axis degree of freedom (e.g., the sliding motion), a horizontal x-axis degree of freedom (e.g., the pulling away motion), a horizontal z-axis degree of freedom (e.g., the booking motion), and/or one or more rotational degrees of freedom (e.g., the rotating motion(s)).

Furthermore, the sternal union and stabilization graft disclosed herein can become semi malleable while maintaining its structural integrity after it has been hydrated. Once implanted, the edges of the incised sternum sections can dig into the graft (e.g., the implant) under the compressive forces introduced through either sternal wire ties or a sternal stabilization plate. This stabilizing effect can cause frictional forces in the midline of the incised sternum to increase significantly. Because of this, the forces that cause instability, gapping and non-union can be diminished by the implant so that the sternum can undergo normal healing.

These functions of the biomechanical stabilizing implant as a biomechanical stabilization tool can be separate from and/or supplementary to other functions as a pain reduction device, a blood loss reduction device, and/or a healing device. For instances, the function as a biomechanical stabilization tool can result from the form factor, semi-malleability, and/or surface area of the implant creating friction fits with the exposed inner areas of the bone sections, and/or being compressed to form a friction-based gasket that locks the two bone sections together. The pain reduction function can result from various medications which can be absorbed into the material of the implant. The blood loss reduction device can also result from various clotting agents absorbed by the material of the implant, and/or by clotting caused by the material pressing against the exposed inner portion of the bones. Furthermore, the healing function can result from the form factor of the implant, the material of the implant, various medications absorbed by the implant, and/or combinations thereof.

The biomechanical stabilizing implant can also improve healing by filling bone voids and integrating into the exposed interiors of the two bone sections as they are compressed together against the biomechanical stabilizing implant. For instance, the fins can extend deep into the exposed cut portion of the sternum. Furthermore, the biomechanical stabilizing implant can operate as a delivery system to distribute drugs and/or biologics deep into the interior of the bone. Moreover, the biomechanical stabilizing implant can operate to absorb fluids, such as blood and saline, to improve clotting at the target location. In other words, in addition to its bone healing qualities, the implant can have structural integrity implications and qualities, such as bone stabilization and immobilization in a 360-degree movement of the thoracic cavity. When augmented with sternal hardware, the biomechanical stabilizing implant can help stabilize compromised bone and can immobilize the sternum with the use of sternal hardware. Additionally, the biomechanical stabilizing implant can be used to augment thoracic skeletal fixation system hardware.

Additional benefits and advantages will become apparent from the detailed disclosure below.

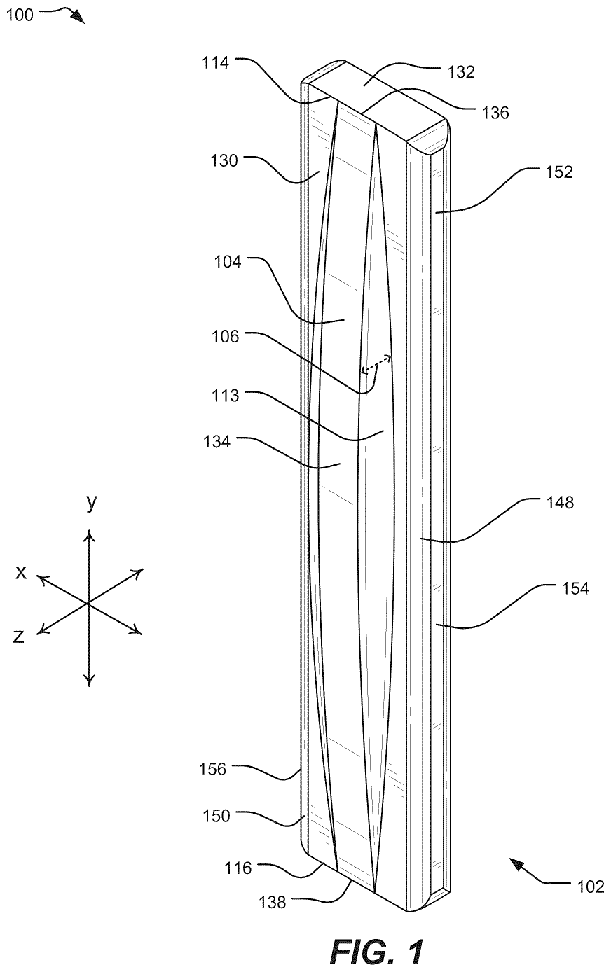

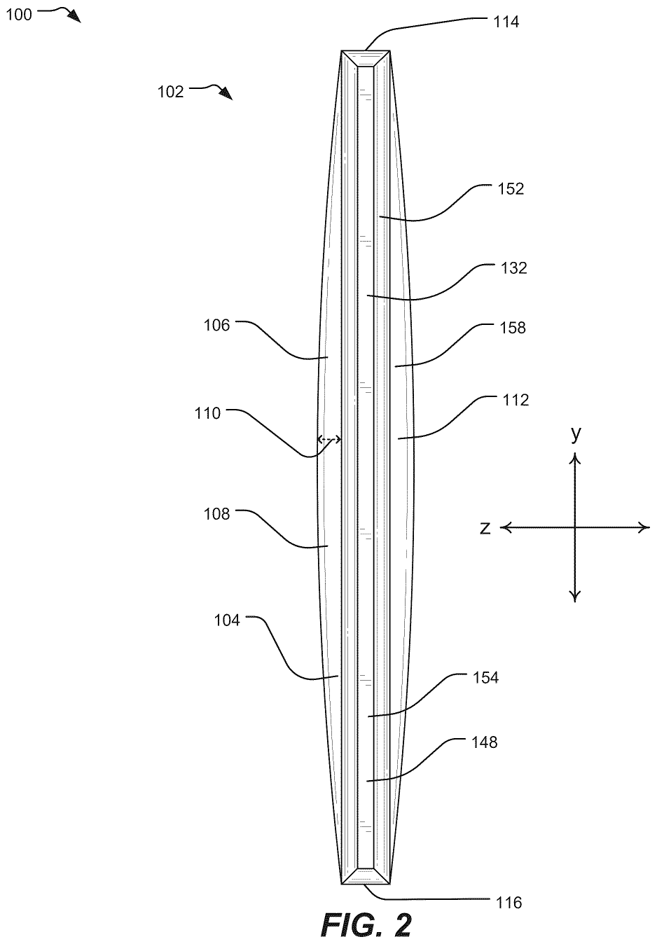

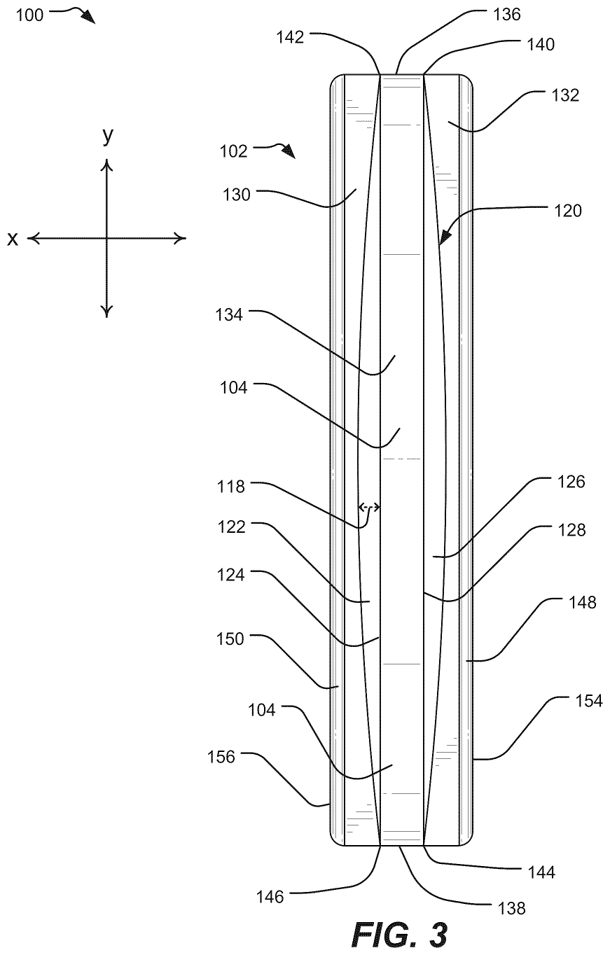

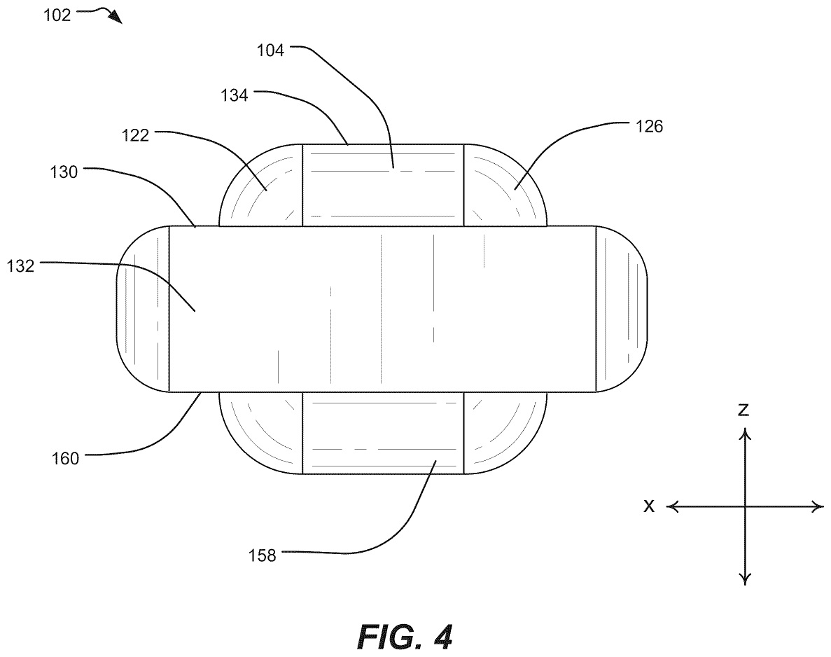

FIGS. 1-4 illustrate an example system 100 including a biomechanical stabilizing implant 102 for placement between two bone portions being rejoined. The biomechanical stabilizing implant 102 can include a first fin 104 extending with a curved depth 106, such that the depth value (e.g., in a z-axis direction) gradually changes to form a curved side profile 108. The curved side profile 108 can have a greatest depth value 110 at a y-axis midpoint 112. Additionally or alternatively, the greatest depth value 110 can be at a location 113 between the y-axis midpoint 112 and a first end 114 of the biomechanical stabilizing implant 102 (e.g., a top end), and/or a second end 116 of the biomechanical stabilizing implant 102 (e.g., a bottom end). In other words, the greatest depth value 110 can be positioned anywhere along the y-axis (e.g., at eighths, sevenths, fifths, quarters, thirds, and/or half points) along the y-axis.

In some instances, the first fin 104 can have a tapered width 118 such that a width value (e.g., in an x-axis direction) gradually changes from the first end 114 to the second end 116, thus forming a curved front profile 120. The curved front profile 120 can include a first elongated taper 122 running along a first side 124 of the first fin 104, and a second elongated taper 126 running along a second side 128 of the first fin 104, which can be symmetrical or asymmetrical to the first elongated taper 122. The first elongated taper 122 and the second elongated taper 126 can form rounded, chamfered corners between the first fin 104 and a first sidewall 130 from which the first fin 104 can extend. For instance, the first sidewall 130 can be a planar side surface of a central body portion 132 of the biomechanical stabilizing implant 102, and the curved or flat surfaces of the first elongated taper 122 and the second elongate taper 126 can extend from an outer surface 134 of the first fin 104 to the first sidewall 130 of the central body portion 132. The first fin 104 can have many different shapes and/or sizes to provide the functionality disclosed herein (e.g., different depth values, different width values, different lengths, flat versus curved, and/or can extend only a portion of the central body portion 132).

In some examples, the outer surface 134 of the first fin 104, and the curved side profile 108 formed by this outer surface 134, can terminate at a first edge 136 of the biomechanical stabilizing implant (e.g., a top edge), and/or at a second edge 138 (e.g., a lower edge) of the biomechanical stabilizing implant 102. Moreover, the tapered width 118 of the first fin 104 can also terminate at the first edge 136 and/or the second edge 138, and can intersect the outer surface at a first termination point 140, a second termination point 142, a third termination point 144, and/or a fourth termination point 146. As such, the first elongated taper 122 and the second elongated taper 126, defined by the curved depth 106 and tapered width 118, can also terminate at the first termination point 140, the second termination point 142, the third termination point 144, and/or the fourth termination point 146.

Furthermore, in some examples, the central body portion 132 can include a tapered front 148 and/or a tapered back 150. The tapered front 148 and/or the tapered back 150 can include a tapering depth value (e.g., in the x-direction) such that the leading edges of the biomechanical stabilizing implant 102 have generally elongated trapezoidal prism-shaped profiles 152. In other words, the front surface 154 and/or the back surface 156 of the biomechanical stabilizing implant 102 can have narrower depth values and/or lesser length values than the central body portion 132.

In some scenarios, the biomechanical stabilizing implant 102 can include a second fin 158, extending from a second sidewall 160 of the central body portion 132, which can be similar to and/or identical to the first fin 104, such that the first fin 104 and/or the second fin 158 give the biomechanical stabilizing implant a symmetrical profile. Additionally or alternatively, the second fin 158 can have a different shape than the first fin 104, such as their respective greatest depth value(s) 110 are different and/or at different locations in the y-axis direction. The fins 104 and 158 can also have different tapered width 118 dimension(s). Furthermore, the first fin 104 and/or the second fin 158 can have a length of the curved side profiles 108 which extends from the first end 114 to the second end 116 (e.g., a full length of the biomechanical stabilizing implant 102 in the y-axis direction). Moreover, in some examples, the first fin 104 and/or the second fin 108 can extend only or at least 90%, 80%, 70%, 60%, 50%, 40%, 30%, 20% or 10% of the length of the biomechanical stabilizing implant 102 in the y-axis direction. Some biomechanical stabilizing implants 102 can include a single first fin 104 extending from the first sidewall and/or a single second fin 158 extending from the second sidewall 160. These singular fins can be centrally positioned along the length of the biomechanical stabilizing implant 102 (e.g., with the location 113 of the greatest depth value 110 at the y-axis midpoint 112), and/or can extend a majority and/or an entire length of the biomechanical stabilizing implant 102. Also, rather than singular fins, the first fin 104 and/or the second fin 158 can include a plurality of fins forming one or more rows of fins along the first sidewall and/or the second side wall 160.

In some examples, a particular fin profile shape feature, such as the location 113 of the greatest depth value 110 and/or the various tapers and curves disclosed herein, can correspond to a particular bone shape of a target bone being healed with the biomechanical stabilizing implant 102. For instance, the location of the greatest depth value 110 can correspond to a widened or deeper portion of the target bone, such as a particular location on the sternum. In this way, the curved side profile 108 can cause the biomechanical stabilizing implant 102 to be especially effective at securing to and healing the bone. By way of example, the biomechanical stabilizing implant 102 can have a width dimension of between 0.8 cm and 1.8 cm (e.g., 1.2 cm, 1.3 cm, 1.4 cm, etc.). The biomechanical stabilizing implant 102 can have a depth dimension of between 0.4 cm and 1.2 cm (e.g., 0.7 cm, 0.8 cm, 0.9 cm, etc.). Furthermore, the biomechanical stabilizing implant 102 can have a length dimension of between 5.0 cm and 9.0 cm (e.g., 6.0 cm, 7.0 cm, 8.0 cm, etc.) In this way, the biomechanical stabilizing implant 102 can have a width dimension, a depth dimension, and/or a length dimension sized for placement between two portions of sternum bone (e.g., a left half of the sternum and the right half of the sternum). Furthermore, the various disclosed components of the biomechanical stabilizing implant 102 can have many different shapes and sizes while maintaining the functionalities discussed herein.

In some instances, the biomechanical stabilizing implant 102 can be formed of one or more of: demineralized bone matrix (DBM), cortical bone fibers, cancellous bone fibers, collagen sponge, cortical bone graft, synthetic bone, tissue graft, and/or combinations thereof. For instance, the biomechanical stabilizing implant 102 can include a collagen matrix derived from bovine, porcine, plant fibers, and/or any combination thereof. The collagen matrix can be embedded in a center of the biomechanical stabilizing implant 102 (e.g., forming the central body portion 132) and/or the collagen matrix can form the first fin 104 and/or the second fin 158. Moreover, this collagen lattice matrix can be designed to provide additional structural integrity to the body of the biomechanical stabilizing implant 102 to give it increased strength and structural integrity with sheer forces and other natural thoracic body movement forces as they interact with the biomechanical stabilizing implant 102 after it has been implanted.

In some scenarios, the biomechanical stabilizing implant 102 can also include one or more carrier agents such as a cellular bone growth factor, antibiotics, analgesics, medications, adhesives, delivery vehicles, and/or combinations thereof. For instance, the bone growth factor can include bone morphogenetic proteins, mesenchymal stem cells, blood, osteoclasts, osteoblasts, hydroxyapatite (HA), exosomes, and/or combinations thereof. The adhesives can include a mollusk-based adhesive. The delivery vehicles can include one or more of bioglass, ceramics, and/or ceramics-derivatives. Furthermore, the biomechanical stabilizing implant 102 can be formed of and/or can include as a carrier agent one or more biologics. The methods disclosed herein can further include soaking the implant in the at least one carrier agent the cellular growth factor during the surgical procedure and/or prior to insertion into the bone sections. Additionally, the matrix of the biomechanical stabilizing implant 102 can have a similar or identical composition as surgical mesh or gauze, such as an intertwined fiber crosshatch.

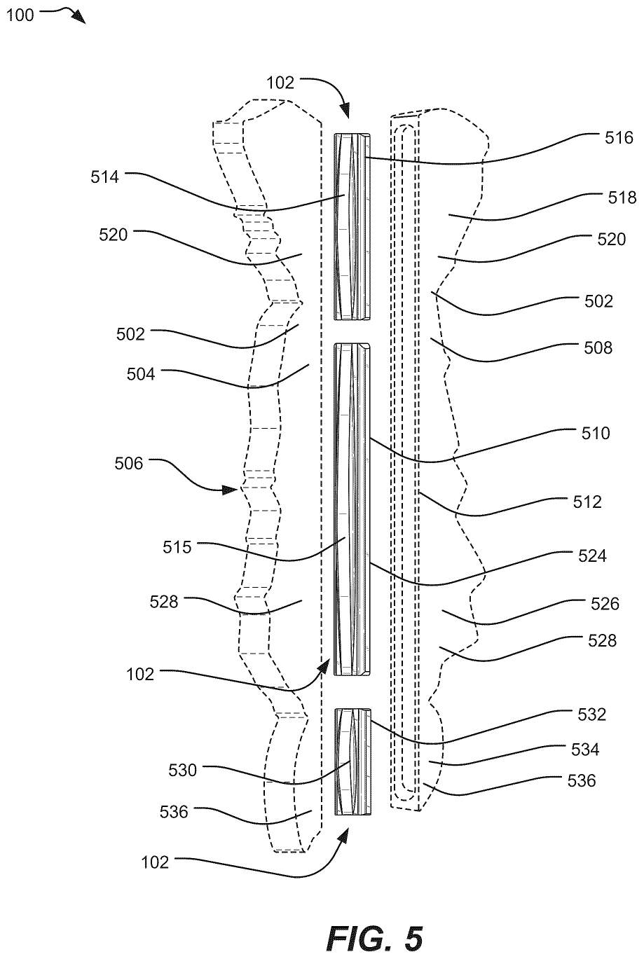

FIG. 5 depicts an example system 100 to heal two portions of bone 502 with one or more biomechanical stabilizing implant(s) 102. For instance, the two portions of bone 502 can include a first section 504 (e.g., a first half) of a sternum 506 and a second section 508 (e.g., a second half) of the sternum 506. In some scenarios, one or more biomechanical stabilizing implant(s) 102 can have a first length dimension 510 which corresponds to a second length dimension 512 of the portion of bone 502. In some scenarios, the system 100 can include a plurality of biomechanical stabilizing implants 102, which can have different first length dimensions 510 corresponding to different second length dimensions 512 of different portions of bone. For instance, a first biomechanical stabilizing implant 514 can have a length dimension 516 corresponding to a dimension of a manubrium 518 for placement between two sections 520 of the manubrium 522. A second biomechanical stabilizing implant 515 can have a length dimension 524 corresponding to a dimensions of a sternum body 526 for placement between two sections 528 of the sternum body. Additionally or alternatively, a third biomechanical stabilizing implant 530 can have a length dimension 532 corresponding to a xiphoid process 534 for placement between two sections 536 of the xiphoid process.

Furthermore, in some examples, a demineralized bone matrix (DBM)-based putty-like additive can be used with the biomechanical stabilizing implant(s) 102. The putty-like additive can have the following characteristics: 1. Flowable and kneadable like dough (handling characteristics). 2. Can be press fit into the surrounding bone, especially in voids as well as the bone marrow or cancellous bone (handling characteristics). 3. IRM (Irrigation Resistant Matrix) can provide hemostatic control can be a first functional quality of the matrix. 4. Insulates severed nerve endings in the sternum and ribs. For instance, the matrix of the putty-like additive can cover over the exposed severed nerve endings and helps prevent ragged bone from grinding against raw, exposed nerve endings. By doing so, the matrix can help significantly cut down on post op pain (second functional quality). 5. Reinforces poor quality bone and bone voids: The putty-like additive can help reinforce poor bone by interdigitating into the pores of the bone as the moldable quality of the matrix allows for it to be pressed into and through the pores and defects of bone. This is part of the functionality of the matrix. 6. Reinforces implant hardware used for sternal closure and rib fixation: The putty-like additive IRM can be used to reinforce, augment, fill gaps, and help stabilize sternal and rib fixation hardware. This is part of the functionality quality of the matrix. 7. Hollow sternum syndrome: The putty-like additive IRM can be used to backfill large bone voids in the sternum. 8. Bone defects in rib fractures: The putty-like additive IRM can be used to augment rib fracture hardware especially where there may be segments of bone loss due to trauma. 9. The healing qualities of the putty-like additive IRM: As a moldable bone grafting matrix, the putty-like additive is formulated to stimulate bone growth over time in all types of bone conditions such as trauma, disease, post op surgical support, hardware augmentation and structurally compromised bone of all types. 10. BioGlass™ (e.g., a surface reactive glass and/or ceramic material that is biocompatible and/or degradable in body fluid) as a growth factor: The putty-like additive (e.g., and/or the biomechanical stabilizing implant(s) 102 absent the putty-like additive) can carry an active cellular growth factor called BioGlass™. BioGlass™ as a cellular growth factor can act like a catalyst that causes the putty-like additive IRM to be both osteoconductive as well as osteoinductive. This means the graft is not only a good scaffold on which bone can grow, but because of the bioglass active cellular matrix, it allows the graft to actively recruit bone growth cells such as osteoblasts and osteoclasts into the graft from the surrounding bone. 11. Healing qualities: as the putty-like additive carries both conductivity and inductivity, this can help speed the healing process of new bone formation. 12. Utility application: the putty-like additive IRM can be directly applied to the affected bone surface by the surgeon. It can be rolled, smeared, press fit, used to augment hardware, used to fill voids, and used to reinforce bone.

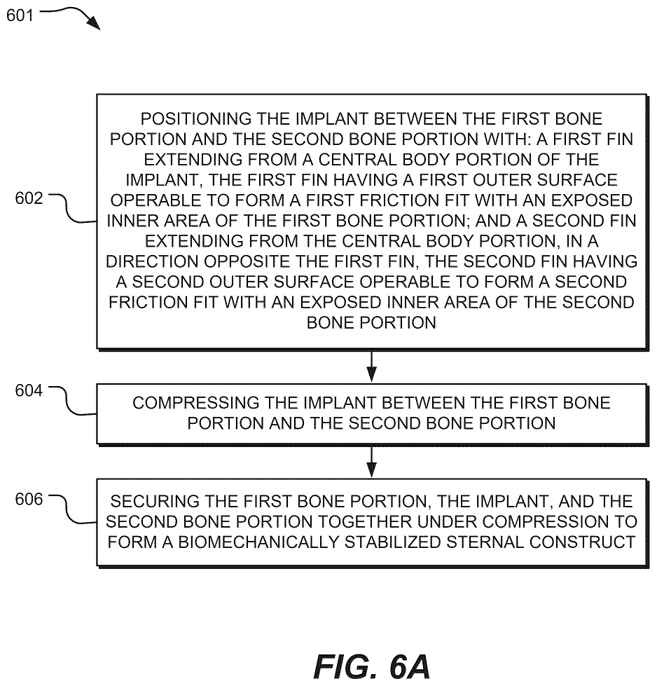

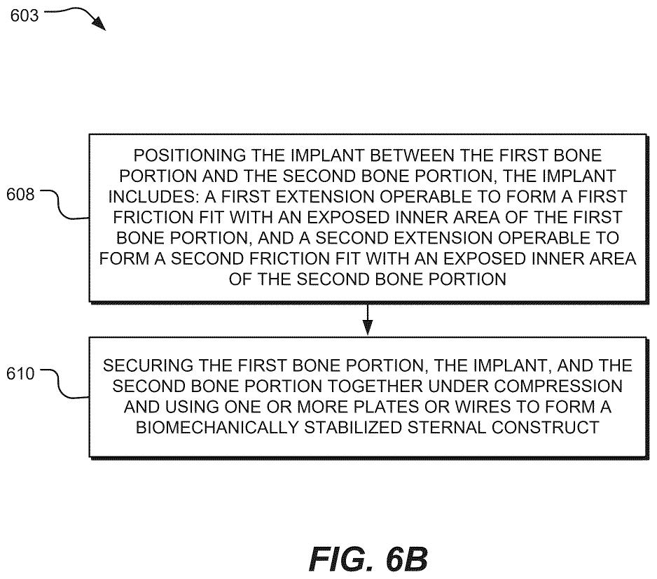

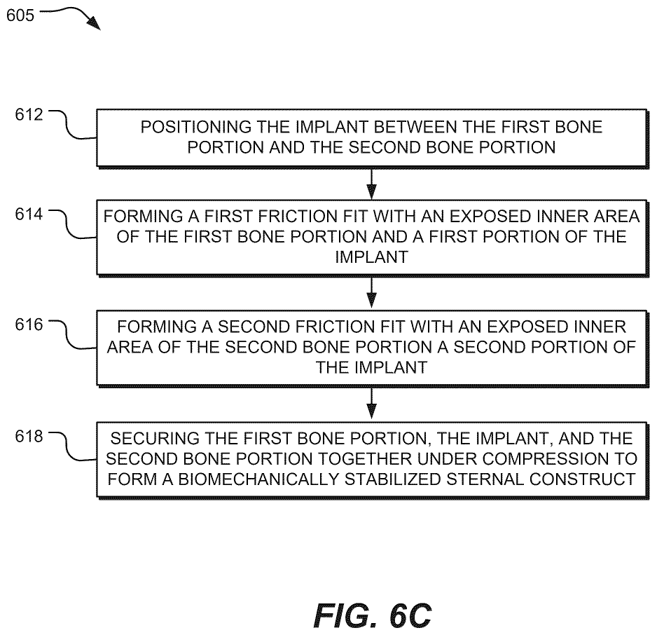

FIGS. 6A-6C depict example method 601, method 603, and method 605 of immobilizing two sections of bone by using a biomechanical stabilizing implant 102. The method(s) 601, 603, and/or 605 can be performed by any of the systems 100 disclosed herein regarding FIGS. 1-5.

In some examples, at operation 602, the method 601 can position the implant between the first bone portion and the second bone portion with: a first fin extending from a central body portion of the implant, the first fin having a first outer surface operable to form a first friction fit with an exposed inner area of the first bone portion; and a second fin extending from the central body portion, in a direction opposite the first fin, the second fin having a second outer surface operable to form a second friction fit with an exposed inner area of the second bone portion. At operation 604, the method 601 can compress the implant between the first bone portion and the second bone portion. At operation 606, the method 601 can secure the first bone portion, the implant, and the second bone portion together under compression to form a biomechanically stabilized sternal construct.

Turning to FIG. 6B, in some instances, at operation 608, the method 603 can position the implant between the first bone portion and the second bone portion, the implant including: a first extension operable to form a first friction fit with an exposed inner area of the first bone portion, and a second extension operable to form a second friction fit with an exposed inner area of the second bone portion. At operation 610, the method 603 can secure the first bone portion, the implant, and the second bone portion together under compression and using one or more plates or wires to form a biomechanically stabilized sternal construct.

Additionally, as shown in FIG. 6C, in some examples, at operation 612, the method 605 can position the implant between the first bone portion and the second bone portion. At operation 614, the method 605 can form a first friction fit with an exposed inner area of the first bone portion and a first portion of the implant. At operation 616, the method 605 can form a second friction fit with an exposed inner area of the second bone portion a second portion of the implant. At operation 618, the method 605 can secure the first bone portion, the implant, and the second bone portion together under compression to form a biomechanically stabilized sternal construct.

It is to be understood that the specific order or hierarchy of steps in the method(s) depicted throughout this disclosure are instances of example approaches and can be rearranged while remaining within the disclosed subject matter. For instance, any of the operations depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A method of biomechanically stabilizing a first bone portion and a second bone portion by using an implant, the method comprising:
positioning the implant between the first bone portion and the second bone portion with:
a first fin extending from a central body portion of the implant, the first fin having a first outer surface operable to form a first friction fit with an exposed inner area of the first bone portion; and
a second fin extending from the central body portion, in a direction opposite the first fin, the second fin having a second outer surface operable to form a second friction fit with an exposed inner area of the second bone portion;
compressing the implant between the first bone portion and the second bone portion; and
securing the first bone portion, the implant, and the second bone portion together under compression to form a biomechanically stabilized sternal construct.

2. The method of claim 1,
wherein,
the biomechanically stabilized sternal construct uses the first friction fit and the second friction fit to form a friction-based gasket preventing movement of the first bone portion relative to the second bone portion.

3. The method of claim 2,
wherein,
the movement prevented by the friction-based gasket includes movement caused by a plurality of different forces.

4. The method of claim 3,
wherein,
the plurality of different forces includes a shear force.

5. The method of claim 4,
wherein,
the friction-based gasket prevents a sliding movement, caused by the shear force, of the first bone portion relative to the second bone portion.

6. The method of claim 3,
wherein,
the plurality of different forces includes a torque force.

7. The method of claim 6,
wherein,
the friction-based gasket prevents a rotational movement, caused by the torque force, of the first bone portion relative to the second bone portion.

8. The method of claim 3,
wherein,
the plurality of different forces includes a tensional force.

9. The method of claim 8,
wherein,
the friction-based gasket prevents a pulling away movement, caused by the tensional force, of the first bone portion relative to the second bone portion.

10. The method of claim 3,
wherein,
the plurality of different forces includes a force caused by an expansion or contraction of lungs of a patient receiving the implant.

11. The method of claim 10,
wherein,
the friction-based gasket prevents a booking motion of the first bone portion relative to the second bone portion caused by the expansion or the contraction of the lungs.

12. The method of claim 1,
wherein,
the biomechanically stabilized sternal construct includes at least one of a plate or a wire used to secure the first bone portion, the implant, and the second bone portion together.

13. A method of biomechanically stabilizing a first bone portion and a second bone portion by using an implant, the method comprising:
positioning the implant between the first bone portion and the second bone portion, the implant includes:
a first extension operable to form a first friction fit with an exposed inner area of the first bone portion, and
a second extension operable to form a second friction fit with an exposed inner area of the second bone portion; and
securing the first bone portion, the implant, and the second bone portion together under compression and using one or more plates or wires to form a biomechanically stabilized sternal construct.

14. The method of claim 13,
wherein,
the implant provides at least dual functions as a biomechanical stabilization tool and a healing tool.

15. The method of claim 13,
wherein,
the implant provides at least dual functions as a biomechanical stabilization tool and a blood loss reduction tool.

16. The method of claim 13,
wherein,
the implant provides at least dual functions as a biomechanical stabilization tool and a pain reduction tool.

17. The method of claim 13, further comprising:
maintaining the compression of the biomechanically stabilized sternal construct with at least one of a wire or a plate coupled to the first bone portion and the second bone portion.

18. A method of biomechanically stabilizing a first bone portion and a second bone portion by using an implant, the method comprising:
positioning the implant between the first bone portion and the second bone portion;
forming a first friction fit with an exposed inner area of the first bone portion and a first portion of the implant; and
forming a second friction fit with an exposed inner area of the second bone portion a second portion of the implant; and
securing the first bone portion, the implant, and the second bone portion together under compression to form a biomechanically stabilized sternal construct.

19. The method of claim 18,
wherein,
the biomechanically stabilized sternal construct prevents movement resulting from a shear force, a torque force, a tension force, and a booking force.

20. The method of claim 18, further comprising:
immobilizing, with the biomechanically stabilized sternal construct, the first bone portion relative to the second bone portion in at least three degrees of freedom.
